(19) United States
(12) Patent Application Publication
(10) Pub. No.: US 2012/0028292 A1
(43) Pub. Date: Feb. 2, 2012

(54) METHODS FOR DIAGNOSING KIDNEY DAMAGE ASSOCIATED WITH HEART FAILURE

(76) Inventors: Georg Hess, Mainz (DE); Andrea Horsch, Mannheim (DE); Dietmar Zdunek, Tutzing (DE)

(21) Appl. No.: 13/267,981

(22) Filed: Oct. 7, 2011

Related U.S. Application Data
(63) Continuation of application No. PCT/EP2010/055868, filed on Apr. 29, 2010.

(30) Foreign Application Priority Data
Apr. 30, 2009 (EP) 09159234.5

(51) Int. Cl. C12Q 1/02; C12M 1/34; G01N 30/00; G01N 33/53

(52) U.S. Cl. 435/29; 436/501; 435/287.1; 422/69

(57) ABSTRACT

Disclosed is a method for diagnosing kidney damage in a subject suffering from heart failure including the steps of a) determining the amounts of liver-type fatty acid binding protein (L-FABP) and kidney injury molecule 1 (KIM-1) and optionally a natriuretic peptide in a sample of a subject, b) forming the L-FABP/KIM-1 ratio, c) comparing the amounts determined in step a) with reference amounts, and diagnosing the kidney damage. Also disclosed are a device and a kit for carrying out the method.

### RELATED APPLICATIONS

This application is a continuation of PCT/EP2010/055868 filed Apr. 29, 2010 and claims priority to EP 09159234.5 filed Apr. 30, 2009.

### FIELD

The present invention relates to diagnostic methods and means. Specifically, it relates to a method for diagnosing kidney damage, preferably chronic kidney damage, more preferably tubular damage and tubular repair, in particular chronic tubular damage and tubular repair, in individuals suffering from heart failure who are in need of a suitable therapy. Moreover, the present invention relates to devices, kits for carrying out said method and a method of deciding on a suitable therapy in patients suffering from heart failure associated kidney damage.

### BACKGROUND

In heart failure (HF), the heart may not provide tissues with adequate blood for metabolic needs, and cardiac-related elevation of pulmonary or systemic venous pressures may result in organ congestion. This condition can result from abnormalities of systolic or diastolic function or, commonly, both.

As cardiac function deteriorates, renal blood flow and GFR decrease, and blood flow within the kidneys is redistributed. The filtration fraction and filtered sodium decrease, but tubular resorption increases, leading to sodium and water retention. Blood flow is further redistributed away from the kidneys during exercise, but renal blood flow improves during rest, possibly contributing to nocturia.

Decreased perfusion of the kidneys activates the renin-angiotensin-aldosterone system, increasing Na and water retention and renal and peripheral vascular tone. These effects are amplified by the intense sympathetic activation accompanying HF.

The renin-angiotensin-aldosterone-vasopressin system produces a cascade of potentially deleterious long-term effects. Angiotensin II worsens HF by causing vasoconstriction, including efferent renal vasoconstriction, and by increasing aldosterone production, which not only enhances Na reabsorption in the distal nephron but also produces myocardial and vascular collagen deposition and fibrosis.

Cardiovascular diseases are increasing with increasing age, so nearly 40% of the population aged 50 already have a detectable cardiovascular disease, which applies for 70% of the population at the age of 75 years (American Heart Association: Heart disease and Stroke statistics—2006, update Dallas AHA 2006, Braunwald Heart disease 8^{th} edition, page 9, FIG. 1-7).

There are manifold causes of the cardiovascular disease which are among others smoking, arterial hypertension, often in connection with metabolic syndrome which is in addition characterized by hyperlipemia, obesity and insulin resistance. Cardiovascular disease may result in heart failure which can be found in 1.5% of all individuals at the age of 50 years and in approximately 10% of individuals at the age of 75 (American Heart Association, Heart Disease and Stroke Statistics 2003, update Dallas AMA 2002).

Heart failure can lead to kidney damage or renal disorder. A first hint for kidney damage is the presence of protein in urine (micro- or macroalbuminuria) which can be assessed by simple dip stick. The most common test for renal disorders used to date is still creatinine while acknowledging its missing accuracy.

Early identification of kidney damage in subjects suffering from heart failure is highly desirable.

Renal function can be assessed by means of the glomerular filtration rate (GFR). For example, the GFR may be calculated by the Cockgroft-Gault or the MDRD formula (Levey 1999, Annals of Internal Medicine, 461-470). GFR is the volume of fluid filtered from the renal glomerular capillaries into the Bowman's capsule per unit time. Clinically, this is often used to determine renal function. The GFR was originally estimated (the GFR can never be determined, all calculations derived from formulas such as the Cockgroft Gault formula of the MDRD formula deliver only estimates and not the “real” GFR) by injecting inulin into the plasma. Since inulin is not reabsorbed by the kidney after glomerular filtration, its rate of excretion is directly proportional to the rate of filtration of water and solutes across the glomerular filter. In clinical practice however, creatinine clearance is used to measure GFR. Creatinine is an endogenous molecule, synthesized in the body, which is freely filtered by the glomerulus (but also secreted by the renal tubules in very small amounts). Creatinine clearance (CrCl) is therefore a close approximation of the GFR. The GFR is typically recorded in millilitres per minute (mL/min). The normal range of GFR for males is 97 to 137 mL/min, the normal range of GFR for females is 88 to 128 mL/min.

GFR is indicative of the kidneys' capacity of water and solutes filtration. A decreased GFR occurs in case of loss of renal tissue (e.g., by necrotic processes). GFR is not indicative for certain renal disorders, e.g., tubular damage. Tubular damage may be present even when GFR is normal.

One of the first hints for kidney damage is the presence of protein in urine (micro- or macroalbuminuria) which can be assessed by simple dip stick. The most common test used to date is still creatinine while acknowledging its missing accuracy.

The studies of Damman et al (Eur. J. of Heart Failure 10 (2008), 997-1000) show that urinary neutrophil gelatinase associated lipocalin (NGAL), a marker of tubular damage, is increased in patients with chronic heart failure (CHF). CHF patients had lower glomerular filtration rates (GFR) and, but higher N terminal-pro brain natriuretic peptide (NT-ProBNP) levels.

Del Vecchio et al (Nature clinical Practice Nephrology 3, (2007), 42-48) reports about the role of aldosterone in kidney damage. Experimental evidence suggests that aldosterone contributes to renal damage. Aldosterone infusion can counteract the beneficial effects of treatment with angiotensin-converting-enzyme (ACE) inhibitors, causing more-severe proteinuria and an increased number of vascular and glomerular lesions, treatment with aldosterone antagonists can reverse these alterations.

Remuzzi et al (Kidney International, Vol. 68, Supplement 99 (2005), S57-S65) studied the role of renin-angiotensin-aldosterone system (RAAS) in the progression of chronic kidney disease. Angiotensin II contributes to accelerate renal damage. ACE inhibitors or angiotensin II receptor antagonists can be used in combination to maximize RAAS inhibition and more effectively reduce proteinurea and GFR decline in diabetic and non-diabetic renal disease. Add-on therapy with an aldosterone antagonist may further increase renoprotection.

According to the study of Kollerits et al there is evidence that adiponectin in blood serum may serve as a gender-specific independent predictor of chronic kidney disease progression associated with the metabolic syndrome (Kollerits et al. (2007), Kidney Int. 71 (12):1279-86). The role of adiponectin in urine was not studied.

Kamijo et al. (Urinary liver-type fatty acid binding protein as a useful biomarker in chronic kidney disease. Mol. Cell Biochem. 2006, 284) reported that urinary excretion of L-FABP may reflect various kind of stresses that cause tubulointerstitial damage and may be a useful clinical marker of the progression of chronic renal disease.

Van Timmeren et al. (J. Pathol 2007, 212:209-217) reported that tubular kidney injury molecule (KIM-1) is upregulated in renal disease and is associated with renal fibrosis and inflammation. Moreover urinary KIM-1 reflects tissue KIM-1, indicating that it can be used as a non-invasive biomarker in renal disease. One advantage of KIM-1 as a urinary biomarker is the fact that its expression seems to be limited to the dysfunctional kidney (P. Devarajan, Expert Opin. Med, Diagn, (2008) 2 (4):387-398).

However, reliable methods for diagnosing kidney damage, in particular tubular damage, in individuals suffering from heart failure who are in need of a suitable therapy have not been described yet.

The technical problem underlying the present invention can be seen as the provision of means and methods for complying with the aforementioned needs. The technical problem is solved by the embodiments characterized in the claims and herein below.

### SUMMARY

Accordingly, the present invention relates to a method of diagnosing kidney damage in a subject with heart failure or suspected to suffer from heart failure, based on the comparison of the amounts of liver-type fatty acid binding protein (L-FABP) or a variant thereof and kidney injury molecule 1 (KIM-1) or a variant thereof, determined in a sample of said subject, preferably determined in a urine sample of the subject, to at least one reference amount.

It is also provides a method for diagnosing kidney damage in a subject with heart failure or suspected to suffer from heart failure, comprising the steps of:
a) determining the amounts of liver-type fatty acid binding protein (L-FABP) or a variant thereof and kidney injury molecule 1 (KIM-1) or a variant thereof in a urinary sample of a subject,
b) comparing the amounts determined in step a) with reference amounts,
c) optionally forming the L-FABP/KIM-1 ratio,
whereby the kidney damage is diagnosed or wherein the comparison of the determined amounts with the reference amounts or the formed L-FABP/KIM-1 ratio is indicative of the patient to suffer from kidney damage

The method of the present invention may comprise the following steps: a) determining the amounts of liver-type fatty acid binding protein (L-FABP) or a variant thereof, preferably urinary liver-type fatty acid binding protein (L-FABP), and kidney injury molecule 1 (KIM-1) or a variant thereof, in a sample, preferably a urine-sample of a subject, b) comparing the amounts determined in step a) with reference amounts.

The diagnosis of the kidney disease may be established based on the information obtained in step b) and preferably based on the information obtained in a) and b).

Accordingly, the present invention relates to a method for diagnosing kidney damage in a subject with heart failure or suspected to suffer from heart failure comprising at least one of the following steps:
a) determining the amounts of liver-type fatty acid binding protein (L-FABP) or a variant thereof and kidney injury molecule 1 (KIM-1) or a variant thereof in a urinary sample of a subject,
b) comparing the amounts determined in step a) with reference amounts, and
c) diagnosing the kidney damage.

In a preferred embodiment of the present invention, the amount of a natriuretic peptide or a variant thereof is determined in a sample of the subject, in general a serum sample. This additional step is preferably carried out when the respective subject is suspected to suffer from heart failure.

In a further preferred embodiment of the present invention, the L-FABP/KIM-1 ratio is formed.

The method of the present invention is, preferably, an in vitro method. Moreover, it may comprise steps in addition to those explicitly mentioned above. For example, further steps may relate to sample pre-treatments or evaluation of the results obtained by the method.

### BRIEF DESCRIPTION OF THE FIGURES

FIG. 1: Plot of L-FABP versus KIM-1 in patients with heart failure. FIG. 1 shows that both markers do not correlate, i.e. the degree of tubular repair does not coincide with tubular damage.

FIG. 2: Plot of NT-pro-BNP versus L-FABP/KIM-1 ratio. FIG. 2 shows that both values correlate to a certain extent, however, individual differences exist meaning that an elevated NT-proBNP value is not mandatorily associated with tubular damage/repair.

FIG. 3: Plot of NT-pro-BNP versus L-FABP for patients with heart failure on ACE inhibitors and a subgroup also on aldosterone antagonists. FIG. 3 shows that tubular damage is lower after administration aldosterone antagonists.

FIG. 4: Plot of NT-pro-BNP versus KIM-1 for patients with heart failure on ACE inhibitors and a subgroup also on aldosterone antagonists.

### DETAILED DESCRIPTION

Diagnosing as used herein refers to assessing the probability according to which a subject suffers from the diseases referred to in this specification. As will be understood by those skilled in the art, such an assessment is usually not intended to be correct for 100% of the subjects to be diagnosed. The term, however, requires that a statistically significant portion of subjects can be diagnosed to suffer from the disease (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001.

Diagnosing as used herein preferably refers to analyzing and where appropriate monitoring of the relevant disease. In particular, diagnosing means analyzing the pathology of specific parts of an organ, e.g., glomerulus and tubulus of the kidney, in particular the tubules and estimating the extent of damage and repair, particular of the tubulus. Monitoring relates to keeping track of the already diagnosed disease, in particular to analyze the progression of the disease or the influence of a particular treatment on the progression of disease. Most preferably, diagnosing relates to analyzing the pathology of tubules in the kidney and estimating the extent of damage and repair in the tubules.

The term “subject” as used herein relates to animals, preferably mammals, and, more preferably, humans. However, it is envisaged by the present invention that the subject shall be suffering or at least is suspected to suffer from heart failure as specified hereinafter. Except for the heart failure and kidney damage, the subject, preferably, shall be apparently healthy, in particular with respect to kidney function (based on the upper limit for serum creatinine).

The terms “kidney damage”, “kidney disease” or “renal disorders” are well known to the person skilled in the art.

In this context, the term “renal disorder” is considered to relate, preferably, to any dysfunction of the kidney or any dysfunction affecting the capacity of the kidney for waste removal and/or ultrafiltration, in particular any impairment of kidney function as determined by methods known to the person skilled in the art, preferably by GFR and/or creatinine clearance. Examples for renal disorders include congenital disorders and acquired disorders. Examples for congenital renal disorders include congenital hydronephrosis, congenital obstruction of urinary tract, duplicated ureter, horseshoe kidney, polycystic kidney disease, renal dysplasia, unilateral small kidney. Examples for acquired renal disorders include diabetic or analgesic nephropathy, glomerulonephritis, hydronephrosis (the enlargement of one or both of the kidneys caused by obstruction of the flow of urine), interstitial nephritis, kidney stones, kidney tumors (e.g., Wilms tumor and renal cell carcinoma), lupus nephritis, minimal change disease, nephrotic syndrome (the glomerulus has been damaged so that a large amount of protein in the blood enters the urine. Other frequent features of the nephrotic syndrome include swelling, low serum albumin, and high cholesterol), pyelonephritis, renal failure (e.g., acute renal failure and chronic renal failure).

In a preferred embodiment of the present invention, the terms “kidney damage” and “kidney disease” exclude any dysfunction of the kidney or any dysfunction affecting the capacity of the kidney for waste removal and/or ultrafiltration, in particular any impairment of kidney function as determined by methods known to the person skilled in the art, preferably by GFR and/or creatinine clearance. The terms in particular exclude congenital hydronephrosis, congenital obstruction of urinary tract, duplicated ureter, horseshoe kidney, polycystic kidney disease, renal dysplasia, unilateral small kidney, diabetic or analgesic nephropathy, glomerulonephritis, hydronephrosis, interstitial nephritis, kidney stones, kidney tumors (e.g., Wilms tumor and renal cell carcinoma), lupus nephritis, minimal change disease, nephrotic syndrome (swelling, low serum albumin, and high cholesterol), pyelonephritis, renal failure, in particular acute kidney injury (acute renal failure) and chronic kidney disease (chronic renal failure) and cardiorenal syndrome. The terms “kidney damage” and “kidney disease” in particular refer to tubular damage optionally associated with tubular repair. Tubular damage, optionally associated with tubular repair, is also referred to as “progressive tubular disease” in the context of the present invention. As in the context of the present invention subjects which suffer from heart failure or are suspected to suffer from heart failure are diagnosed, tubular damage and/or tubular repair are also referred to as “heart failure associated kidney damage”.

Renal disorders can be diagnosed by means known to the person skilled in the art. Particularly, renal function (which is used interchangeably with “kidney function” in the context of the present invention) can be assessed by means of the glomerular filtration rate (GFR). For example, the GFR may be calculated by the Cockgroft-Gault or the MDRD formula (Levey 1999, Annals of Internal Medicine, 461-470). GFR is the volume of fluid filtered from the renal glomerular capillaries into the Bowman's capsule per unit time. Clinically, this is often used to determine renal function. The GFR was originally estimated (the GFR can never be determined, all calculations derived from formulas such as the Cockgroft Gault formula of the MDRD formula deliver only estimates and not the “real” GFR) by injecting inulin into the plasma. Since inulin is not reabsorbed by the kidney after glomerular filtration, its rate of excretion is directly proportional to the rate of filtration of water and solutes across the glomerular filter. In clinical practice however, creatinine clearance is used to measure GFR. Creatinine is an endogenous molecule, synthesized in the body, which is freely filtered by the glomerulus (but also secreted by the renal tubules in very small amounts). Creatinine clearance (CrCl) is therefore a close approximation of the GFR. The GFR is typically recorded in millilitres per minute (mL/min). The normal range of GFR for males is 97 to 137 mL/min, the normal range of GFR for females is 88 to 128 mL/min.

GFR is indicative of the kidneys' capacity of water and solutes filtration. A decreased GFR occurs in case of loss of renal tissue (e.g., by necrotic processes). GFR is not indicative for certain renal disorders, e.g., tubular damage. Tubular damage may be present even when GFR is normal.

One of the first hints for renal disorder is the presence of protein in urine (micro- or macroalbuminuria) which can be assessed by simple dip stick. The most common test used to date is still creatinine while acknowledging its missing accuracy.

Chronic kidney disease (CKD) and acute kidney injury (AKI) are known to the person skilled in the art and generally recognized as referring to renal failure as determined by GFR or creatinine clearance.

CKD is known as a loss of renal function which may worsen over a period of months or even years. The symptoms of worsening renal function are unspecific. In CKD glomerular filtration rate is significantly reduced, resulting in a decreased capability of the kidneys to excrete waste products by water and solute filtration. Creatinine levels may be normal in the early stages of CKD. CKD is not reversible. The severity of CKD is classified in five stages, with stage 1 being the mildest and usually causing few symptoms. Stage 5 constitutes a severe illness including poor life expectancy and is also referred to as end-stage renal disease (ESRD), chronic kidney failure (CKF) or chronic renal failure (CRF).

Acute kidney injury (AKI), previously also referred to as acute renal failure (ARF), is a rapid loss of kidney function which may originate from various reasons, including low blood volume and exposure to toxins. Contrary to CKD, AKI can be reversible. AKI is diagnosed on the basis of creatinine levels, urinary indices like blood urea nitrogen (BUN), occurrence of urinary sediment, but also on clinical history. A progressive daily rise in serum creatinine is considered diagnostic of ARF.

The term “cardiorenal syndrome” (also “CRS”) as used in the context of the present invention is to be understood in the sense of the definition established by Ronco et al, in Intensive Care Med. 2008, 34, pages 957-962 and in J. Am. Coll. Cardiol. 2008, 52, p. 1527-1539. Accordingly, CRS refers, in the broadest sense, to a pathophysiologic disorder of the heart and kidneys whereby acute or chronic dysfunction of one of the cited organs may induce acute or chronic dysfunction of the other. The simplest description of CRS is that a relatively normal kidney is dysfunctional because of a diseased heart, assuming that in the presence of a healthy heart, the same kidney would perform normally. 5 subtypes of CRS exist. Type 1 CRS reflects an abrupt worsening of cardiac function (e.g., acute cardiogenic shock or decompensated congestive heart failure) leading to acute kidney injury. Type 2 CRS comprises chronic abnormalities in cardiac function (e.g., chronic congestive heart failure) causing progressive chronic kidney disease. Type 3 CRS consists of an abrupt worsening of renal function (e.g., acute kidney ischemia or glomerulonephritis) causing acute cardiac dysfunction (e.g., heart failure, arrhythmia, ischemia). Type 4 CRS describes a state of chronic kidney disease (e.g., chronic glomerular disease) contributing to decreased cardiac function, cardiac hypertrophy, and/or increased risk of adverse cardiovascular events.

In the context of the present invention, the term “tubular damage” refers to epithelial injury in tubule cells as a consequence of heart failure. The present invention preferably refers to chronic tubular damage. It is believed that in tubular damage tubule cells are ischemic following heart failure, but it is also believed that tubules have retained their functionality within the kidney entirely or at least to the greatest or a great part. This means that renal function is not impaired or only impaired to a lesser extent, such that CKD or AKI will not or cannot be diagnosed by the methods known in the art, i.e. GFR and/or creatinine clearance. In tubular damage, tubule cells may become dysfunctional, in general by necrosis, and die. However, tubular epithelium regeneration is possible after ischemia and even after necrosis, referred to as “tubular repair” in the context of the present invention. As the present invention preferably refers to chronic tubular injury, it likewise refers to chronic tubular repair or tubular repair from chronic tubular damage.

In the context of the present invention, the term “apparently healthy” is known to the person skilled in the art and refers to a subject which does not show obvious signs of an underlying renal disorder. The disorder here is an impaired kidney function, in particular in respect to GFR, for example based on creatinine clearance, in particular its upper limit. The subject, thus, may suffer from an impaired kidney function as defined beforehand, but does not show obvious signs such that the impaired kidney function cannot be diagnosed or assessed without detailed diagnostic examination by a physician. In particular, the diagnosis by a specialist (here: a nephrologist) would be required to diagnose impaired kidney function in the apparently healthy subject not showing obvious symptoms of the disease.

The term “apparently healthy” as used in the context of the present invention, accordingly, is restricted to individuals not showing obvious signs of an impaired kidney function (i.e. of a dysfunction of the kidney) or not having an impaired kidney function (i.e. of a dysfunction of the kidney). An apparently healthy individual, as understood in the context of the present invention, may however suffer from one or more pathophysiological states of the kidney in which kidney function is not impaired, or in which kidney function is not impaired at the onset of the respective disease but which may lead an impaired kidney function. The individual may suffer from microalbuminuria, albuminuria and/or proteinuria and/or any pathophysiological state associated therewith. The individual may also suffer from glomerular damage and/or any pathophysiological state associated therewith. These pathophysiological states are known to the person skilled in the art and include disease associated with glomerular syndromes, preferably: acute nephritic syndromes, in particular glomerulonephritis, nephropathy, nephrotic syndromes, in particular minimal change disease, glomerulosclerosis, glomerulonephritis, diabetic nephropathy, and glomerular vascular syndromes, in particular atherosclerotic nephropathy, hypertensive nephropathy.

The term “heart failure” as used herein relates to an impaired systolic and/or diastolic function of the heart. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest. Heart failure, i.e., an impaired systolic and/or diastolic function of the heart, can be determined also by, for example, echocardiography, angiography, szintigraphy, or magnetic resonance imaging. This functional impairment can be accompanied by symptoms of heart failure as outlined above (NYHA class II-IV), although some patients may present without significant symptoms (NYHA I). Moreover, heart failure is also apparent by a reduced left ventricular ejection fraction (LVEF). More preferably, heart failure as used herein is accompanied by a left ventricular ejection fraction (LVEF) of less than 60%, of 40% to 60% or of less than 40%.

The term “liver-type fatty acid binding protein” (L-FABP, frequently also referred to as FABP1 herein also referred to as liver fatty acid binding protein) relates to a polypeptide being a liver type fatty acid binding protein and to a variant thereof. Liver-type fatty acid binding protein is an intracellular carrier protein of free fatty acids that is expressed in the proximal tubules of the human kidney. For a sequence of human L-FABP, see, e.g., Chan et al.: Human liver fatty acid binding protein cDNA and amino acid sequence, Functional and evolutionary implications, J. Biol. Chem. 260 (5), 2629-2632 (1985) or GenBank Acc. Number M10617.1.

As L-FABP is preferably determined in a urine sample of the respective subject, is may also be referred to, in the context of the present invention, as “urinary liver-type fatty acid binding protein” or “urinary” L-FABP.

The term “L-FABP” encompasses also variants of L-FABP, preferably, of human L-FABP. Such variants have at least the same essential biological and immunological properties as L-FABP, i.e. they bind free fatty acids and/or cholesterol and/or retinoids, and/or are involved in intracellular lipid transport. In particular, they share the same essential biological and immunological properties if they are detectable by the same specific assays referred to in this specification, e.g., by ELISA Assays using polyclonal or monoclonal antibodies specifically recognizing the L-FABP. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the human L-FABP, preferably over the entire length of human L-FABP. How to determine the degree of identity is specified elsewhere herein. Variants may be allelic variants or any other species specific homologs, paralogs, or orthologs. Moreover, the variants referred to herein include fragments of L-FABP or the aforementioned types of variants as long as these fragments have the essential immunological and biological properties as referred to above. Such fragments may be, e.g., degradation products of the L-FABP. Further included are variants which differ due to posttranslational modifications such as phosphorylation or myristylation. The term “L-FABP”, preferably, does not include heart FABP, brain FABP and intestine FABP.

The term “natriuretic peptide” comprises atrial natriuretic peptide (ANP)-type and brain natriuretic peptide (BNP)-type peptides and variants thereof having the same predictive potential. Natriuretic peptides according to the present invention comprise ANP-type and BNP-type peptides and variants thereof (see, e.g., Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP). ANP and BNP have a vasodilatory effect and cause excretion of water and sodium via the urinary tract. Preferably, natriuretic peptides according to the present invention are NT-proANP, ANP, and, more preferably, NT-proBNP, BNP, and variants thereof. ANP and BNP are the active hormones and have a shorter half-life than their respective inactive counterparts, NT-proANP and NT-proBNP. BNP is metabolised in the blood, whereas NT-proBNP circulates in the blood as an intact molecule and as such is eliminated renally. The in-vivo half-life of NTproBNP is 120 min longer than that of BNP, which is 20 min (Smith 2000, J Endocrinol. 167: 239-46). Preanalytics are more robust with NT-proBNP allowing easy transportation of the sample to a central laboratory (Mueller 2004, Clin Chem Lab Med 42: 942-4). Blood samples can be stored at room temperature for several days or may be mailed or shipped without recovery loss. In contrast, storage of BNP for 48 hours at room temperature or at 4° Celsius leads to a concentration loss of at least 20% (Mueller loc.cit., Wu 2004, Clin Chem 50: 867-73). Therefore, depending on the time-course or properties of interest, either measurement of the active or the inactive forms of the natriuretic peptide can be advantageous.

The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc. cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein. The NT-proBNP referred to in accordance with the present invention further encompasses allelic and other variants of said specific sequence for human NT-proBNP discussed above. Specifically, envisaged are variant polypeptides which are on the amino acid level preferably, at least 50%, 60%, 70%, 80%, 85%, 90%, 92%, 95%, 97%, 98%, or 99% identical, to human NT-proBNP, preferably over the entire length of human NT-proBNP. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human NT-proBNP as long as the polypeptides have NT-proBNP properties. NT-proBNP properties as referred to herein are immunological and/or biological properties. Preferably, the NT-proBNP variants have immunological properties (i.e. epitope composition) comparable to those of NT-proBNP. Thus, the variants shall be recognizable by the aforementioned means or ligands used for determination of the amount of the natriuretic peptides. Biological and/or immunological NT-proBNP properties can be detected by the assay described in Karl et al. (Karl 1999, Scand J Clin Invest 230:177-181), Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide.

The term “kidney injury molecule-1” (KIM-1) relates to a type 1 membrane protein containing a unique six-cysteine Ig domain and a mucin domain in its extracellular portion. KIM-1 which is the sequence of rat 3-2 cDNA contains an open reading frame of 307 amino acids.

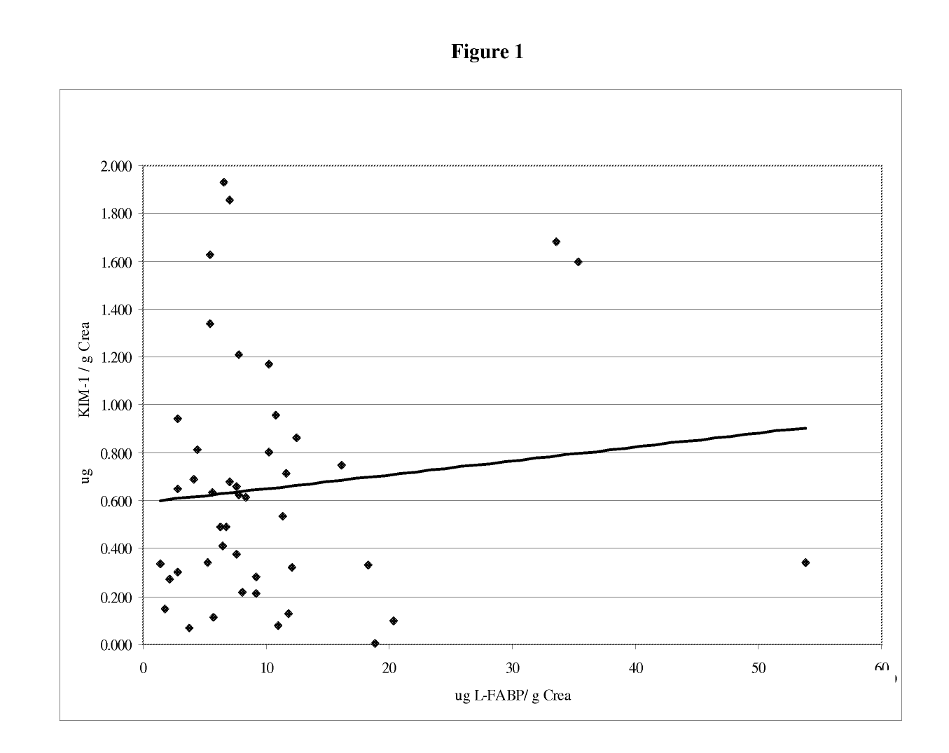

The protein sequence of human cDNA clone 85 also contains one Ig, mucin, transmembrane, and cytoplasmic domain each as rat KIM-1. All six cysteines within the Ig domains of both proteins are conserved. Within the Ig domain, the rat KIM-1 and human cDNA clone 85 exhibit 68.3% similarity in the protein level. The mucin domain is longer, and the cycloplasmic domain is shorter in clone 85 than rat KIM-1, with similarity of 49.3 and 34.8% respectively. Clone 85 is referred to as human KIM-1 (for the structure of KIM-1 proteins see, e.g., Ichimura et al., J Biol Cem, 273 (7), 4135-4142 (1998), in particular FIG. 1). Recombinant human KIM-1 exhibits no cross-reactivity or interference to recombinant rat- or mouse-KIM-1.

KIM-1 mRNA and protein are expressed in high levels in regenerating proximal tubule epithelial cells which cells are known to repair and regenerate the damaged region in the postischemic kidney. KIM-1 is an epithelial cell adhesion molecule (CAM) up-regulated in the cells, which are dedifferentiated and undergoing replication after renal epithelial injury.

A proteolytically processed domain of KIM-1 is easily detected in the urine soon after acute kidney injury (AKI) so that KIM-1 performs as an acute kidney injury urinary biomarker (Expert Opin. Med. Diagn. (2008) 2 (4): 387-398).

KIM-1 referred to in accordance with the present invention further encompasses allelic and other variants of said specific sequence for human KIM-1 discussed above. Specifically, envisaged are variant polypeptides which are on the amino acid level preferably, at least 50%, 60%, 70%, 80%, 85%, 90%, 92%, 95%, 97%, 98%, or 99% identical, to human KIM-1, preferably over the entire length of human KIM-1. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human KIM-1 as long as the polypeptides have KIM-1 properties. “KIM-1 properties” as used in the context of the present invention refers to inducing dedifferentiation and replication after renal epithelial injury.

Determining the amount of L-FABP or a variant thereof, KIM-1 or a variant thereof or a natriuretic peptide or a variant thereof, or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR analyzers, or chromatography devices. Further, methods include micro-plate ELISA-based methods, fully-automated or robotic immunoassays (available for example on ELECSYS analyzers), CBA (an enzymatic cobalt binding assay, available for example on Roche-Hitachi analyzers), and latex agglutination assays (available for example on Roche-Hitachi analyzers).

Preferably, determining the amount of a peptide or polypeptide comprises the steps of (α) contacting a cell capable of eliciting a cellular response the intensity of which is indicative of the amount of the peptide or polypeptide with the peptide or polypeptide for an adequate period of time, (β) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprise the steps of (α) contacting the peptide with a specific ligand, (optionally) removing non-bound ligand, (β) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-tag, glutathione-S-transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include, e.g., horseradish peroxidase, alkaline phosphatase, beta-galactosidase, luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF™ (Amersham Biosciences). A suitable enzyme-substrate combination may result in a colored reaction product, fluorescence or chemiluminescence, which can be measured according to methods known in the art (e.g., using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available, e.g., from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoroimmunoassay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a peptide or polypeptide may be, also preferably, determined as follows: (α) contacting a solid support comprising a ligand for the peptide or polypeptide as specified above with a sample comprising the peptide or polypeptide and (β) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20 (1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “sample” refers to a sample of a body fluid, to a sample of separated cells or to a sample from a tissue or an organ. Samples of body fluids can be obtained by well-known techniques and include, preferably, samples of blood, plasma, serum, urine, samples of blood, plasma or serum. It is to be understood that the sample depends on the marker to be determined. Therefore, it is encompassed that the polypeptides as referred to herein are determined in different samples. L-FABP or a variant thereof and KIM-1 or a variant thereof are preferably determined in a urine sample. Natriuretic peptides or variants thereof are, preferably, determined in a blood serum or blood plasma sample.

The term “forming a ratio” as used herein means calculating in each individual a ratio between the determined amounts of the specified peptides. All ratios were used to calculate medians and respective percentiles to obtain reference kidney disease information for the target disease.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample or a ratio of amounts is compared to a reference ratio of amounts. The comparison referred to in step (c) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format.

In general, for determining the respective amounts/amounts or amount ratios allowing to establish the desired diagnosis in accordance with the respective embodiment of the present invention, (“threshold”, “reference amount”), the amount(s)/amount(s) or amount ratios of the respective peptide or peptides are determined in appropriate patient groups. According to the diagnosis to be established, the patient group may, for example, comprise only healthy individuals, or may comprise healthy individuals and individuals suffering from the pathophysiological (state which is to be determined, or may comprise only individuals suffering from the pathophysiological state which is to be determined, or may comprise individuals suffering from the various pathophysiological states to be distinguished, by the respective marker(s) using validated analytical methods. The results which are obtained are collected and analyzed by statistical methods known to the person skilled in the art. The obtained threshold values are then established in accordance with the desired probability of suffering from the disease and linked to the particular threshold value. For example, it may be useful to choose the median value, the 60th, 70th, 80th, 90th, 95th or even the 99th percentile of the healthy and/or non-healthy patient collective, in order to establish the threshold value(s), reference value(s) or amount ratios.

A reference value of a diagnostic marker can be established, and the amount of the marker in a patient sample can simply be compared to the reference value. The sensitivity and specificity of a diagnostic and/or prognostic test depends on more than just the analytical “quality” of the test-they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker of the invention, a distribution of marker amounts for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al, Radiology 143: 29-36 (1982).

In certain embodiments, markers and/or marker panels are selected to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and most preferably at least about 95%. The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups, a value greater than 1 indicates that a positive result is more likely in the diseased group, and a value less than 1 indicates that a positive result is more likely in the control group. In the case of a negative likelihood ratio, a value of 1 indicates that a negative result is equally likely among subjects in both the “diseased” and “control” groups, a value greater than 1 indicates that a negative result is more likely in the test group, and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups, a value greater than 1 indicates that a positive result is more likely in the diseased group, and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups, a value greater than 1 indicates that the risk is greater in the diseased group, and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

While exemplary panels are described herein, one or more markers may be replaced, added, or subtracted from these exemplary panels while still providing clinically useful results. Panels may comprise both specific markers of a disease (e.g., markers that are increased or decreased in bacterial infection, but not in other disease states) and/or non-specific markers (e.g., markers that are increased or decreased due to inflammation, regardless of the cause, markers that are increased or decreased due to changes in hemostasis, regardless of the cause, etc.). While certain markers may not individually be definitive in the methods described herein, a particular “fingerprint” pattern of changes may, in effect, act as a specific indicator of disease state. As discussed above, that pattern of changes may be obtained from a single sample, or may optionally consider temporal changes in one or more members of the panel (or temporal changes in a panel response value).

The term “reference amounts” as used herein in this embodiment of the invention refers to amounts of the polypeptides which allow diagnosing kidney damage in a subject with heart failure or suspected to suffer from heart failure (in general, this subject is apparently healthy in respect to kidney function).

Therefore, the reference amounts will in general be derived from subjects known to be physiologically healthy, or subjects known to suffer from kidney damage (which may be apparently healthy in respect to kidney function), or subjects suffering from heart failure, or subjects suffering from heart failure and known to suffer from kidney damage.

Accordingly, the term “reference amount” as used herein either refers to an amount which allows diagnosing kidney damage in a subject with heart failure or suspected to suffer from heart failure (in general, this subject is apparently healthy in respect to kidney function). The comparison with reference amounts permits to differentiate between these individuals and those suspected to suffer from heart failure (in general, this subject is apparently healthy in respect to kidney function), but not suffering from kidney damage. In the present invention, “reference amount” also refers to the ratio L-FABP/KIM-1.

Reference amounts for L-FABP or a variant thereof and KIM-1 or a variant thereof may be derived from subjects as defined above in the present invention which suffer from heart failure or are suspected to suffer from heart failure (in which, preferably, are apparently healthy in respect to kidney function), and where the subject was diagnosed to suffer from kidney damage, preferably tubular kidney damage and tubular kidney repair, in particular chronic tubular kidney damage and tubular kidney repair. The amounts of the respective peptide serving for establishing the reference amounts can be determined prior to the diagnosis established in accordance with the present invention.

In all embodiments of the present invention, the amount/amounts of the respective markers used therein (L-FABP or a variant thereof and KIM-1 or a variant thereof) are determined by methods known to the person skilled in the art.

In order to test if a chosen reference value yields a sufficiently safe diagnosis of patients suffering from the disease of interest, one may for example determine the efficiency (E) of the methods of the invention for a given reference value using the following formula:

E=(TP/TO)×100,

wherein TP=true positives and TO=total number of tests=TP+FP+FN+TN, wherein FP=false positives, FN=false negatives and TN=true negatives. E has the following range of values: 0<E<100). Preferably, a tested reference value yields a sufficiently safe diagnosis provided the value of E is at least about 50, more preferably at least about 60, more preferably at least about 70, more preferably at least about 80, more preferably at least about 90, more preferably at least about 95, more preferably at least about 98.

The diagnosis if individuals are healthy or suffer from a certain pathophysiological state is made by established methods known to the person skilled in the art. The methods differ in respect to the individual pathophysiological state.

The algorithms to establish the desired diagnosis are laid out in the present application, in the passages referring to the respective embodiment, to which reference is made.

Accordingly, the present invention also comprises a method of determining the threshold amount indicative for a physiological and/or a pathological state and/or a certain pathological state, comprising the steps of determining in appropriate patient groups the amounts of the appropriate marker(s), collecting the data and analyzing the data by statistical methods and establishing the threshold values.

The term “about” as used herein refers to +/−20%, preferably +/−10%, preferably, +/−5% of a given measurement or value.

The term “reference amount” as used herein refers to an amount which allows diagnosing kidney damage.

It is to be understood that if a reference from a subject is used which suffers from a disease or combination of diseases, an amount of a peptide or protein in a sample of a test subject being essentially identical to said reference amount shall be indicative for the respective disease or combination of diseases. The reference amount applicable for an individual subject may vary depending on various physiological parameters such as age, gender, or subpopulation. Moreover, the reference amounts, preferably define thresholds. Thus, a suitable reference amount may be determined by the method of the present invention from a reference sample to be analyzed together, i.e. simultaneously or subsequently, with the test sample. A suitable technique may be to determine the median of the population for the peptide or polypeptide amounts to be determined in the method of the present invention.

KIM-1 and L-FABP are urinary biomarkers which are increased expressed in the proximal tubule epithelial cells in the postischemic kidney. As L-FABP is considered a biomarker of tubular damage and KIM-1 is believed an indicator of tubular repair, the ratio of both markers reflects evidence of disease progression. Natriuretic peptides, in particular NT-pro-BNP, are considered as biomarkers of heart failure. Natriuretic peptides, in particular NT-pro-BNP, are released during hemodynamic stress. Natriuretic peptides are cleared by the kidneys, and the hypervolemia and hypertension characteristic of renal failure enhance the secretion and elevate the levels of especially NT-pro-BNP.

Therefore, determination of said markers discloses relevant information of pathogenic kidney processes.

Based on the comparison of the amounts of L-FABP or a variant thereof, KIM-1 or a variant thereof and, optionally, a natriuretic peptide or a variant thereof, in particular NT-pro-BNP or a variant thereof, and the corresponding reference amounts and the L-FABP/KIM-1 ratio, the extent and progression of the kidney disease of subjects suffering from heart failure can be characterized.

Advantageously, it has been found that the combination of L-FABP or a variant thereof, KIM-1 or a variant thereof and, optionally, NT-pro-BNP or a variant thereof as biomarkers, in particular the amounts of L-FABP or a variant thereof, KIM-1 or a variant thereof and, optionally, NT-pro-BNP or a variant thereof present in a sample of a subject in combination with the amounts of L-FABP and KIM-1 or, in a preferred embodiment, the ratio of the amounts of L-FABP/KIM-1 allow for the characterization of a heart failure associated kidney disease in a reliable and efficient manner. Moreover, it has been found that the concentrations of said biomarkers do not correlate. Thus, each of said biomarkers is statistically independent from each other. Thanks to the present invention, subjects can be more readily and reliably diagnosed and subsequently treated according to the result of the inventive method.

Increased amounts of NT-pro-BNP or a variant thereof in comparison to reference amounts in a serum sample of a subject are indicative for heart failure, i.e. heart failure patients exhibit increased amounts of NT-proBNP. According to the method of the invention, heart failure (which, in an embodiment of the present invention, is indicated by increased amounts of NT-pro-BNP or a variant thereof in serum) go along with increased amounts of L-FABP or a variant thereof and KIM-1 or a variant thereof in comparison to reference amounts measured in a urinary sample of a subject. This indicates that the extent of the tubular damage of the kidney and the associated repair are dependent from the extent of the heart failure.

Moreover, according to the method of the invention it could be found that the L-FABP/KIM-1 ratio increases with increased amounts of NT-pro-BNP or a variant thereof. This indicates that repair decreases with the progression of the heart failure.

Progressive kidney disease will result in end stage renal disease over variable time periods. The diagnosis of end stage renal disease is based on the kidney function (e.g., creatinine value).

Reference amount of >about 300 pg/ml, preferable >about 450 pg/ml, more preferable >about 600 pg/ml, in particular >about 1000 pg/ml, very particular >about 1500 pg/ml, for NT-pro-BNP or a variant thereof are indicative for heart failure, in particular when in connection with elevated amounts of L-FABP or a variant thereof.

Reference amounts of >about 5 μg/g, preferable >about 7.5 μg/g, more preferable >about 10 μg/g, in particular >about 12.5 μg/g creatinine for L-FABP or a variant thereof are indicative for tubular damage.

A reference amount of >about 300 pg/ml, preferable >about 450 pg/ml, more preferable >about 600 pg/ml, in particular >about 1000 pg/ml, very particular >about 1500 pg/ml, for NT-pro-BNP or a variant thereof and a reference amount of >about 5 μg/g, preferable >about 7.5 μg/g, more preferable >about 10 μg/g, in particular >about 12.5 μg/g creatinine for L-FABP or a variant thereof are indicative for a heart failure associated kidney disease, in particular tubular damage associated with heart failure.

An L-FABP/KIM-1 ratio of <about 13.5, preferable <about 11, more preferable <about 8.5 is indicative for predominant repair over tubular damage of the kidney.

An L-FABP/KIM-1 ratio of >about 13.5, preferable >about 20, more preferable >about 30, in particular >40 is indicative for predominant damage over tubular repair of the kidney.

As outlined elsewhere in the present application, L-FABP represents tubular kidney damage and KIM-1 tubular repair. Thus the ratio between L-FABP and KIM-1 reflects the balance between tubular damage and tubular repair, a process which can result in complete recovery in kidney damage. As outlined in the examples a ratio of 13.5 L-FABP/KIM-1 has been identified in patients with heart failure. In case repair predominates damage a progressive kidney disease is unlikely, in case of the opposite a progressive kidney disease needs to be considered.

Thus if tubular damage is predominant over repair (or if the criteria indicating moderate or more particularly severe tubular damage as laid out above are met) this is a call for more frequent monitoring of urinary biomarkers specifically L-FABP and KIM-1 and in addition kidney function markers such as, e.g., creatinine, cystatin C or GFR. In addition there is a need to avoid drugs or interventions that may give rise to additional kidney damage including application of contrast agents. In addition the cardiac medication requires reconsideration in terms of use of ACE inhibitors and ARBs, including their dose, in addition prescription of aldosterone antagonist needs to be considered.

The higher the afore-mentioned reference amounts of NT-pro-BNP or a variant thereof and L-FABP or a variant thereof alone or in combination with an L-FABP/KIM-1 ratio of >about 13.5, preferable >about 20, more preferable >about 30, in particular >40 the more likely is a progressive and severe disease of the kidney, in particular tubular damage.

A reference amount of <about 5.5 μg/g creatinine for L-FABP or a variant thereof and/or an L-FABP/KIM-1 ratio of <about 9 are indicative for no or only minor disease of the kidney, in particular tubular damage.

In particular a reference amount of <about 300 pg/ml for NT-pro-BNP or a variant thereof, a reference amount of <about 5.5 μg/g creatinine for L-FABP or a variant thereof and/or an L-FABP/KIM-1 ratio of <about 9 are indicative for no or only minor disease of the kidney, in particular tubular damage.

A reference amount of >about 5.5 μg/g creatinine for L-FABP or a variant thereof and/or an L-FABP/KIM-1 ratio of >about 9 are indicative for a moderate disease of the kidney, in particular tubular damage.

In particular a reference amount of >about 300 pg/ml for NT-pro-BNP or a variant thereof, a reference amount of >about 5.5 μg/g creatinine for L-FABP or a variant thereof and/or an L-FABP/KIM-1 ratio of >about 9 are indicative for a moderate disease of the kidney, in particular tubular damage.

A reference amount of >about 7.5 μg/g creatinine for L-FABP or a variant thereof and/or an L-FABP/KIM-1 ratio of >about 14 are indicative for a severe disease of the kidney, in particular tubular damage.

In particular a reference amount of >about 600 pg/ml for NT-pro-BNP or a variant thereof, a reference amount of >about 7.5 μg/g creatinine for L-FABP or a variant thereof and/or an L-FABP/KIM-1 ratio of >about 14 are indicative for a severe disease of the kidney, in particular tubular damage.

A reference amount of >about 20 μg/g creatinine for L-FABP or a variant thereof and/or an L-FABP/KIM-1 ratio of >about 37 are indicative for a very severe disease of the kidney, in particular tubular damage.

In particular a reference amount of >about 1700 for NT-pro-BNP or a variant thereof, a reference amount of >about 20 μg/g creatinine for L-FABP or a variant thereof and/or an L-FABP/KIM-1 ratio of >about 37 are indicative for a very severe disease of the kidney, in particular tubular damage.

The present invention also provides a method of deciding, in a subject suffering from heart failure or suspected to suffer from heart failure and, preferably, being apparently healthy in respect to kidney function, on a suitable therapy for heart failure associated kidney disease, based on the comparison of the amounts of liver-type fatty acid binding protein (L-FABP) or a variant thereof and kidney injury molecule 1 (KIM-1) or a variant thereof, determined in a sample of said subject, preferably determined in a urine sample of the subject, to at least one reference amount.

The method of the present invention may comprise the following steps: a) determining the amounts of liver-type fatty acid binding protein (L-FABP) or a variant thereof, preferably urinary liver-type fatty acid binding protein (L-FABP), and kidney injury molecule 1 (KIM-1) or a variant thereof in a sample, preferably a urine-sample of a subject, b) comparing the amounts determined in step a) with reference amounts.

The decision on the suitable therapy may be established based on the information obtained in step b) and preferably based on the information in steps a) and b).

The present invention therefore also provides a method of deciding, in a subject suffering from heart failure associated kidney damage, on a suitable therapy comprising at least one of the following steps:
a) determining the amounts of liver-type fatty acid binding protein (L-FABP) or a variant thereof and kidney injury molecule 1 (KIM-1) or a variant thereof in a urine sample of a subject,
b) comparing the amounts determined in step a) with reference amounts, thereby diagnosing the kidney damage, and
c) deciding on the suitable therapy.

In one embodiment of the present invention, the L-FABP/KIM-1 ratio is formed.

In a further embodiment of the present invention, the individual is apparently healthy in respect to kidney function

In a further embodiment of the present invention, the amount of a natriuretic peptide or a variant thereof is determined in a sample of the subject, in general a serum sample. This additional step is preferably carried out when the respective subject is suspected to suffer from heart failure.

Suitable therapies are the administration of pharmaceuticals which are effective in respect of:
1. inhibition of further progression of kidney disease,
2. heart failure as such (causing the kidney damage)
3. prevention of further kidney damage (in particular in case of a decreased repair process).

Typical pharmaceuticals of category 1 and 2 are among others Angiotensin-converting enzyme (ACE) inhibitors, beta-blockers, angiotensin II receptor blockers (ARB) and/or aldosterone antagonists.

Category 3 encompasses among others the administration of ACE inhibitors applied in high doses, nonsteroidal anti-inflammatory drugs (NSAIDs) and avoiding the use of radio contrast agents.

The afore-mentioned agents are known to a person skilled in the art. Preferred beta blockers are proprenolol, metoprolol, bisoprolol, carvedilol, bucindolol and/or nebivolol. Suitable ACE inhibitors are in particular Enalapril, Captopril, Ramipril and/or Trandolapril. Suitable angiotensin II receptor blockers are in particular Losartan, Valsartan, Irbesartan, Candesartan, Telmisartan and/or Eprosartan.

Suitable aldosterone antagonists are in particular spironolacton or eplerenone.

A preferred therapy of heart failure is to start with ACE inhibitors or ARBs with or without beta-blocker and the later additional administration of aldosterone antagonists (Braunwald's Heart Disease, 8th edition, D. L. Mann, p. 616, FIG. 25-6).

The afore-mentioned therapies are in particular effective if combined with each other.

As outlined beforehand, an L-FABP/KIM-1 ratio exceeding 13.5 is indicative of excess tubular damage over repair and indicative of progressive kidney damage over time, specifically the higher the ratio can be found, the higher is the assumed risk of progression, specifically if the ratio exceeds 20, 30 and specifically 40. In this case, administration of aldosterone antagonists are to be taken into consideration, in particular if the ratio exceeds 30 or 40. Additionally, drugs or interventions associated with the risk of additive kidney damage are to be avoided. Vice versa ratio below 13.5 indicates that the kidney damage is associated with appropriate repair specifically if the ratio is below 11 or 8 indicating that the kidney damage is unlikely to progress (to progressive kidney damage). In this case, aldosterone antagonists are not required. Moreover other drugs and interventions known to be associated with kidney damage are not contraindicated but still require careful consideration.

The terms “suitable therapy” and “susceptible” as used herein means that a therapy applied to a subject will inhibit or ameliorate the progression of heart failure or its accompanying symptoms and/or of kidney damage or its accompanying symptoms. It is to be understood assessment for susceptibility for the therapy will not be correct for all (100%) of the investigated subjects. However, it is envisaged that at least a statistically significant portion can be determined for which the therapy can be successfully applied. Whether a portion is statistically significant can be determined by techniques specified elsewhere herein.

The present invention also relates to a method of monitoring kidney damage in a subject suffering from heart failure, based on the comparison of the amounts of liver-type fatty acid binding protein (L-FABP) or a variant thereof and kidney injury molecule 1 (KIM-1) or a variant thereof, determined in a sample of said subject, preferably determined in a urine sample of the subject, to at least one reference amount, and repeating the comparison step.

In a preferred embodiment, the above method of monitoring comprises monitoring the therapy.

The method of the present invention may comprise the following steps: a) determining the amounts of liver-type fatty acid binding protein (L-FABP) or a variant thereof, and kidney injury molecule 1 (KIM-1) or a variant thereof in a sample, preferably a urine-sample of a subject, b) comparing the amounts determined in step a) with reference amounts.

Diagnosis of the kidney disease may be established based on the information obtained in step b) and preferably based on the information obtained in a) and b), and monitoring is carried out by repeating step b, preferably by repeating steps a) and b) during therapy.

Accordingly, the present invention relates to a method for monitoring kidney damage in a subject suffering from heart failure comprising at least one of the steps of:
a) determining the amounts of liver-type fatty acid binding protein (L-FABP) or a variant thereof and kidney injury molecule 1 (KIM-1) or a variant thereof in a sample of a subject,
b) comparing the amounts determined in step a) with reference amounts and diagnosing the kidney damage, and
c) repeating steps a) and b) during the therapy.

In one embodiment of the present invention, the L-FABP/KIM-1 ratio is formed. In a further embodiment of the present invention, the method includes deciding on the suitable therapy, after step b), in the case of therapy monitoring.

In an embodiment of the present invention, the amount of a natriuretic peptide or a variant thereof is determined in a sample of the subject, preferably a serum sample. This additional step is preferably carried out when the respective subject is suspected to suffer from heart failure.

Monitoring relates to keeping track of the already diagnosed disease, in particular to analyze the progression of the disease or the influence of a particular treatment on the progression of disease. Monitoring means control preferably after 2 weeks, more preferably after 1 month, most preferably after 3, 6 or 12 months, depending on the state as clinically needed.

As outlined above the necessity of monitoring is associated with the suspected progression of the kidney damage, in particular tubular damage, or the assessment of drugs and interventions affecting kidney damage, in particular tubular damage. For example if the L-FABP/KIM-1 ratio exceeds 13.5 or even 20, 30 or 40 monitoring within 3, 2 or 1 months is preferred, if the ratio of L-FABP/KIM-1 is below 13.5, 11 or 8 monitoring at 6 to 12 months interval is sufficient. If medicaments have been applied that may affect the kidney monitoring after 2 weeks or 1 month is preferred.

Accordingly, the present invention relates to a method for diagnosing myocardial infarction in a subject comprising at least one of the following steps:
a) determining the amounts of a natriuretic peptide and/or troponin T in a sample of the subject,
b) comparing the amounts determined in step a) with reference amounts, and
c) diagnosing myocardial infarction.

Moreover, the present invention also envisages kits and devices adapted to carry out the method of the present invention.

Furthermore, the present invention relates to a device for diagnosing kidney damage in a subject with heart failure or suspected to suffer from heart failure comprising:
a) means for determining the amounts of liver-type fatty acid binding protein (L-FABP) or a variant thereof and kidney injury molecule 1 (KIM-1) or a variant thereof in a urinary sample of a subject,
b) means for comparing the amounts determined in step a) with reference amounts,
whereby the device is adapted for diagnosing the kidney damage.

In a preferred embodiment of the present invention, the device furthermore comprises means for forming the L-FABP/KIM-1 ratio.

The sample, preferably, is a urinary sample.

In an embodiment of the present invention, the device furthermore comprises means for determining the amounts of a natriuretic peptide in a serum sample of a subject, and/or means for comparing the amounts determined with reference amounts, and optionally means for diagnosing the suspected disease,

The term “device” as used herein relates to a system of means comprising at least the aforementioned means operatively linked to each other as to allow the differentiation. Preferred means for determining the amount of a one of the aforementioned polypeptides as well as means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the polypeptides in an applied sample and a computer unit for processing the resulting data for the evaluation. The computer unit, preferably, comprises a database including the stored reference amounts or values thereof recited elsewhere in this specification as well as a computer-implemented algorithm for carrying out a comparison of the determined amounts for the polypeptides with the stored reference amounts of the database. Computer-implemented as used herein refers to a computer-readable program code tangibly included into the computer unit. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the polypeptides referred to herein, Plasmon surface resonance devices, NMR spectrometers, mass-spectrometers etc.) and/or evaluation units/devices referred to above in accordance with the method of the invention.

Moreover the present invention is concerned with a kit for diagnosing kidney damage in a subject with heart failure or suspected to suffer from heart failure comprising:
a) means for determining the amounts of liver-type fatty acid binding protein (L-FABP) or a variant thereof and kidney injury molecule 1 (KIM-1) or a variant thereof in a urinary sample of a subject,
b) means for comparing the amounts determined in step a) with reference amounts,
whereby the kit is adapted for diagnosing the kidney damage.

In a preferred embodiment of the present invention, the kit furthermore comprises means for forming the L-FABP/KIM-1 ratio.

The sample, preferably, is a urinary sample.

In an embodiment of the present invention, the kit furthermore comprises means for determining the amounts of a natriuretic peptide in a serum sample of a subject, and/or means for comparing the amounts determined with reference amounts, and optionally means for diagnosing the suspected disease,

The term “kit” as used herein refers to a collection of the aforementioned compounds, means or reagents of the present invention which may or may not be packaged together. The components of the kit may be comprised by separate vials (i.e. as a kit of separate parts) or provided in a single vial. Moreover, it is to be understood that the kit of the present invention is to be used for practicing the methods referred to herein above. It is, preferably, envisaged that all components are provided in a ready-to-use manner for practicing the methods referred to above. Further, the kit preferably contains instructions for carrying out the methods. The instructions can be provided by a user's manual in paper- or electronic form. For example, the manual may comprise instructions for interpreting the results obtained when carrying out the aforementioned methods using the kit of the present invention.

How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the KIM-1, L-FABP and a cardiac troponin. Plasmon surface resonance devices, NMR spectrometers, mass-spectrometers etc.) or evaluation units/devices referred to above in accordance with the method of the invention.

The present invention also relates to the use of a kit or device for determining the amount of KIM-1 or a variant thereof, L-FABP or a variant thereof and optionally a natriuretic peptide or a variant thereof in a sample of a subject, comprising means for determining the amount of KIM-1, L-FABP and optionally a natriuretic peptide and/or means for comparing the amount of KIM-1, L-FABP and optionally a natriuretic peptide to at least one reference amount for: diagnosing kidney damage in a subject with heart failure or suspected to suffer from heart failure and being apparently healthy in respect to kidney function, and/or deciding whether a subject suffering or suspected to suffer from heart failure associated kidney damage and being apparently healthy in respect to kidney function is susceptible to a suitable therapy, and/or monitoring kidney damage in a subject suffering from heart failure associated kidney damage.

The present invention also relates to the use of: an antibody against KIM-1 or a variant thereof, an antibody against L-FABP or a variant thereof and optionally an antibody against a natriuretic peptide or a variant thereof, and/or of means for determining the amount of KIM-1 or a variant thereof, L-FABP or a variant thereof and optionally a natriuretic peptide or a variant thereof, and/or of means for comparing the amount of KIM-1 or a variant thereof, L-FABP or a variant thereof and optionally a natriuretic peptide or a variant thereof to at least one reference amount for the manufacture of a diagnostic composition for: diagnosing kidney damage in a subject with heart failure or suspected to suffer from heart failure and preferably being apparently healthy in respect to kidney function, and/or deciding whether a subject suffering or suspected to suffer from heart failure associated kidney damage and being apparently healthy in respect to kidney function is susceptible to a suitable therapy, and/or monitoring kidney damage in a subject suffering from heart failure associated kidney damage.

The present invention also relates to the use of: an antibody against KIM-1 or a variant thereof, an antibody against L-FABP or a variant thereof and optionally an antibody against a natriuretic peptide or a variant thereof, and/or of means for determining the amount of KIM-1 or a variant thereof, L-FABP or a variant thereof and optionally a natriuretic peptide or a variant thereof and/or of means for comparing the amount of KIM-1 or a variant thereof, L-FABP or a variant thereof and optionally a natriuretic peptide or a variant thereof to at least one reference amount for: diagnosing kidney damage in a subject with heart failure or suspected to suffer from heart failure and being apparently healthy in respect to kidney function, and/or deciding whether a subject suffering or suspected to suffer from heart failure associated kidney damage and being apparently healthy in respect to kidney function is susceptible to a suitable therapy, and/or monitoring kidney damage in a subject suffering from heart failure associated kidney damage.

All references cited in this specification are herewith incorporated by reference with respect to their entire disclosure content and the disclosure content specifically mentioned in this specification.

The following examples shall merely illustrate the invention. They shall not be construed, whatsoever, to limit the scope of the invention.

### Example 1

Patients suffering from systolic heart failure (a total of 44 patients: LVEF <40%, normal kidney function based on the upper limit for serum creatinine) were investigated for urine levels of KIM-1 and L-FABP and serum levels of NT-pro-BNP. The patients did not suffer from cardiorenal syndrome, i.e. they did not suffer from any form of cardiorenal syndrome (acute and chronic cardiorenal syndrome, acute and chronic renocardiac syndrome, secondary cardiorenal syndrome). For the definition of cardiorenal syndrome and its various forms reference is made to Ronco et al, Intensive Care Med (2208), 34:957-962.

The patients (clinically stable) were subjected to different anti-inflammatory therapies over at least 4 weeks:
Group 1: treatment with ACE inhibitors alone
Group 2: treatment with ACE inhibitors in combination with spironolactone, an aldosterone antagonist

The levels of said biomarkers were determined using the following commercially available immunoassay kits:

Urinary levels of said biomarkers were determined using the following commercially available immunoassay kits:

L-FABP was determined by using the L-FABP ELISA-Kit from CMIC Co., Ltd, Japan. The test is based on an ELISA 2-step assay. L-FABP standard or urine samples are firstly treated with pretreatment solution, and transferred into an anti-L-FABP antibody coated microplate containing assay buffer and incubated. During this incubation, L-FABP in the reaction solution binds to the immobilized antibody. After washing, the 2nd antibody-peroxidase conjugate is added as the secondary antibody and incubated, thereby forming a complex of the L-FABP antigen sandwiched between the immobilized antibody and the conjugate antibody. After incubation, the plate is washed and substrate for enzyme reaction is added, color develops according to the L-FABP antigen quantity. The L-FABP concentration is determined based on the optical density.

Human KIM-1 was determined by the Human KIM-1 (catalogue number DY 1750) ELISA Development kit from R&D-Systems, containing a capture antibody (goat anti-human KIM-1) and a detection antibody (biotinylated goat anti-human KIM-1). A seven point standard curve using 2-fold serial dilutions in Reagent Diluent, and a high standard of 2000 pg/mL is recommended.

Serum levels of NT-proBP were determined by the ELECSYS proBNP II assay from Roche Diagnostics

The biomarker concentrations of said study and the L-FABP/KIM-1 ratio are summarized in the following table.

TABLE 1
Biomarker Concentrations in Patients with Heart Failure

| | NT-pro-BNP | u-L-FABP | KIM-1 μg/g | L-FABP/ |
|---|---|---|---|---|
| Percentile | pg/ml | μg/g Creatinine | Creatinine | KIM-1 Ratio |
| 50^{th} median | 600 | 7.66 | 0.57 | 13.80 |
| 25th | 322 | 5.42 | 0.31 | 8.74 |
| 75th | 1793 | 11.45 | 0.84 | 36.88 |

TABLE 2
Urinary biomarkers classified by NT-pro-BNP < > Median

| | 244 pg/ml | 1547 pg/ml |
|---|---|---|
| NT-pro-BNP | < median = 600 pg/ml | > median = 600 pg/ml |
| L-FABP [μg/ml | 6.62 | 9.20 |
| creatinine] | | |
| KIM-1 [μg/ml creatinine] | 0.34 | 0.75 |

Table 2 shows that with elevated levels of NT-pro-BNP in serum, the amounts of urinary L-FABP and KIM-1 are increased as well. This indicates that the extent of the tubulary damage of the kidney and the associated repair are dependent from the extent of the heart failure.

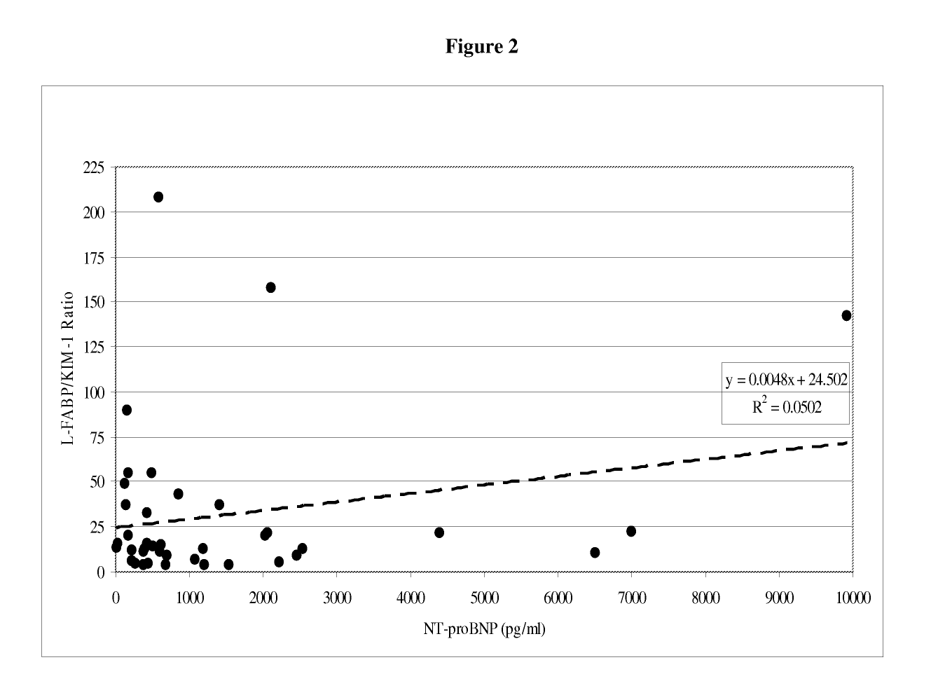

FIG. 2 shows that the L-FABP/KIM-1 ratio increases with increased amounts of NT-pro-BNP. This indicates that repair decreases with the progression of the heart failure.

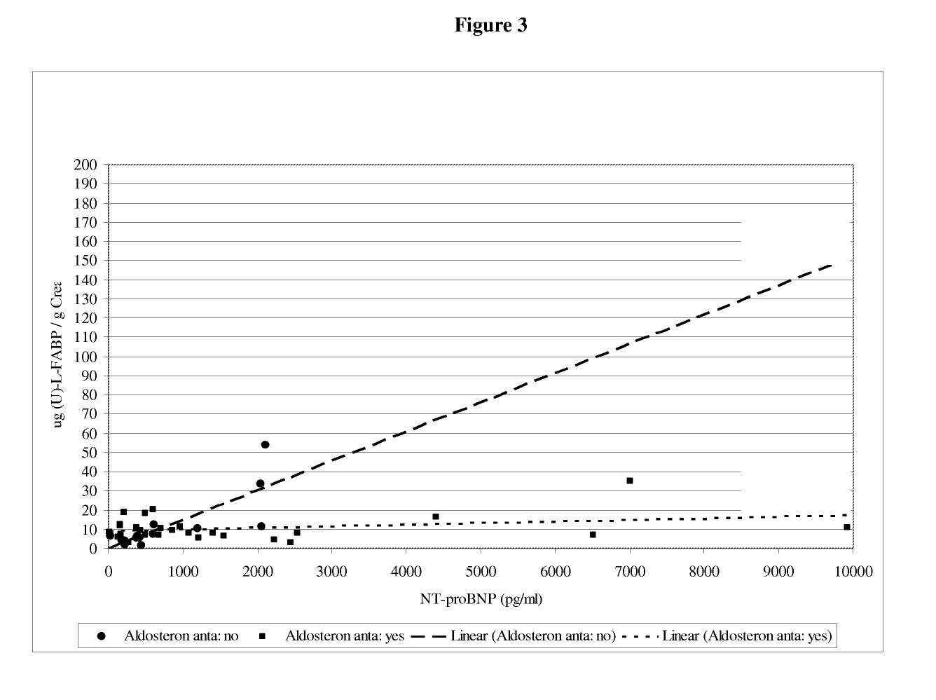

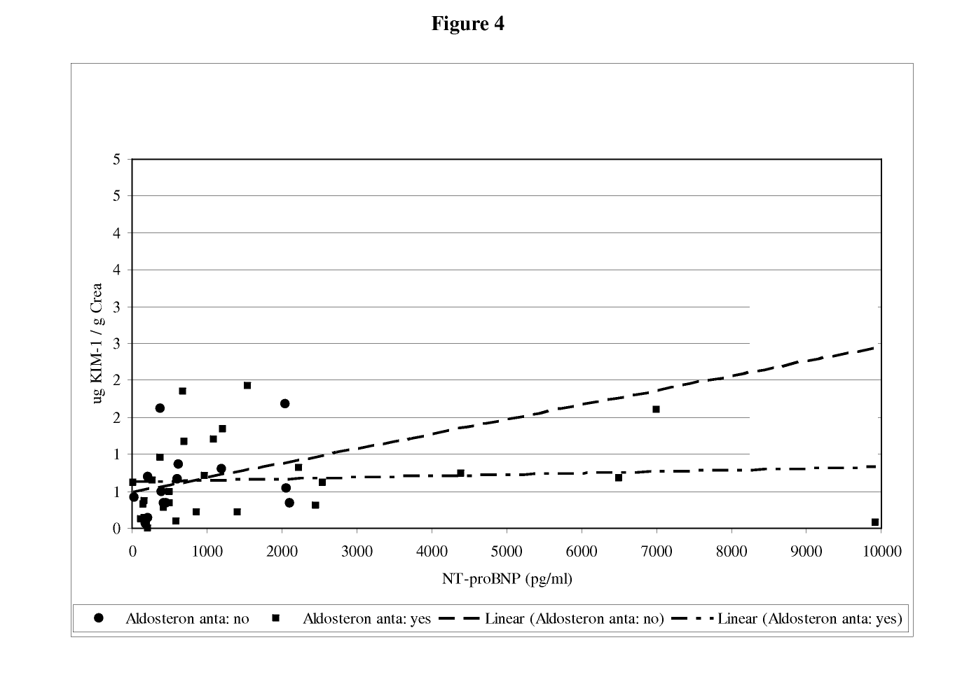

The administration of spironolactone leads to a regression of the tubular damage of the kidney (see FIG. 3) and to decreased tubulary repair (see FIG. 4). Therefore, patients with elevated L-FABP and KIM-1 levels will benefit from an additional spironolactone therapy. In particular, patients with significantly increased levels of NT-pro-BNP will benefit from said therapy.

### Example 2

A total of 64 patients without clinical evidence of heart failure, who underwent coronary angiography including STENT implantation and, thus, were at increased risk of overt heart failure, were tested for L-FABP and KIM-1. They were 41 males and 23 females (mean age 62.3 years). Median NT-pro BNP was found to be 397 pg/ml (134 pg/ml and 1220 pg/ml for the 25th and 75th percentile). Since all patients did not have overt heart failure none of the patients was on treatment with aldosterone antagonists, whereas all patients were given ACE inhibitors. The patients did not suffer from any form of cardiorenal syndrome (acute and chronic cardiorenal syndrome, acute and chronic renocardiac syndrome, secondary cardiorenal syndrome). For the definition of cardiorenal syndrome and its various forms reference is made to Ronco et al, Intensive Care Med (2208), 34:957-962.

Urine and plasma samples were obtained before angiography and STENT implantation, all patients were clinically stable within the last 3 weeks, kidney function was in the normal range in all patients as indicated by creatinine levels within normal.

Blood was centrifuged within 30 minutes and the resulting serum was kept at −20° C. until tested. Urine samples were also kept in aliquots at −20° C. until tested.

Tests were done as previously described.

### Results:

| | FABP | L-KIM-1 | L-FABP/KIM-1 | |
|---|---|---|---|---|
| Percentile | (pg/ml) | (pg/ml) | (pg/ml) | NT-proBNP (pg/ml) |
| 25 | 3.8 | 0.277 | 6.8 | 134 |
| 50 | 6.8 | 0.56 | 13.2 | 397 |
| 75 | 12.2 | 0.75 | 26.1 | 1220 |

### Conclusion:

Patients with documented coronary artery disease but without evidence of overt heart failure (but impaired cardiac function) had L-FABP and KIM-1 levels in the range of those with overt heart failure and moderately elevated NT-pro BNP Levels (NT-pro BNP below 600 pg/ml).

This shows that impaired cardiac function in individuals is associated with a risk to suffer from kidney damage. Such patients may benefit from aldosterone antagonist therapy, as do heart failure patients. In contrast to the previous understanding in the field, such patients may benefit from aldosterone antagonist therapy, as do heart failure patients.

What is claimed is:

1. A method for diagnosing kidney damage in a subject with heart failure or suspected to suffer from heart failure, the method comprising the steps of:
determining an amount of liver-type fatty acid binding protein (L-FABP) and an amount of kidney injury molecule 1 (KIM-1) in a urine sample from the subject,
comparing the amounts of L-FABP and KIM-1 determined with reference amounts of L-FABP and KIM-1,
calculating an L-FABP/KIM-1 ratio from the amounts determined and comparing the calculated ratio with a reference L-FABP/KIM-1 ratio, and
diagnosing the kidney damage, wherein an increased amount of L-FABP compared to the reference amount of L-FABP and a decreased amount of KIM-1 compared to the reference amount of KIM-1, resulting in a high value of the L-FABP/KIM-1 ratio compared to the reference L-FABP/KIM-1 ratio, are indicative for progressive tubular damage of the kidney.

2. A method for diagnosing kidney damage in a subject with heart failure or suspected to suffer from heart failure, the method comprising the steps of:
determining an amount of liver-type fatty acid binding protein (L-FABP) and an amount of kidney injury molecule 1 (KIM-1) in a urine sample from the subject,
comparing the amounts of L-FABP and KIM-1 determined with reference amounts of L-FABP and KIM-1,
calculating an L-FABP/KIM-1 ratio from the amounts determined and comparing the calculated ratio with a reference L-FABP/KIM-1 ratio,
determining an amount of N-terminal pro brain natriuretic peptide (NT-proBNP) in a serum sample from the subject,
comparing the amount of NT-proBNP determined with a reference amount of NT-proBNP,
diagnosing the kidney damage, wherein an increased L-FABP/KIM-1 ratio compared to the reference L-FABP/KIM-1 ratio and an increased amount of NT-pro-BNP compared to the reference amount of NT-proBNP indicates progressive tubular disease.

3. The method according to claim 2, wherein the reference amount for NT-pro-BNP is selected from the group consisting of >about 300 pg/ml, >about 450 pg/ml, and >about 600 pg/ml, and the reference amount for L-FABP is selected from the group consisting of >about 5 μg/g creatinine, >about 7.5 μg/g creatinine, and >about 10 μg/g creatinine.

4. The method according to claim 1, wherein an L-FABP/KIM-1 ratio selected from the group consisting of <about 13.5, <about 11, and <about 8.5 is indicative for predominant repair over tubular damage of the kidney.

5. The method according to claim 1, wherein an L-FABP/KIM-1 ratio selected from the group consisting of >about 13.5, >about 20, >about 30, and >about 40 is indicative for predominant damage over tubular repair of the kidney.

6. A method for deciding whether a subject suffering from heart failure associated kidney damage is susceptible to a suitable therapy, the method comprising the steps of:
determining an amount of liver-type fatty acid binding protein (L-FABP) and an amount of kidney injury molecule 1 (KIM-1) in a urine sample from the subject,
comparing the amounts of L-FABP and KIM-1 determined with reference amounts of L-FABP and KIM-1,
calculating an L-FABP/KIM-1 ratio from the amounts determined and comparing the calculated ratio with a reference L-FABP/KIM-1 ratio,
determining an amount of N-terminal pro brain natriuretic peptide (NT-proBNP) in a serum sample from the subject,
comparing the amount of NT-proBNP determined with a reference amount of NT-proBNP, and
diagnosing the kidney damage from the comparisons made and deciding on the suitable therapy.

7. A method for monitoring kidney damage in a subject suffering from heart failure or suspected to suffer from heart failure, the method comprising the steps of:
determining an amount of liver-type fatty acid binding protein (L-FABP) and an amount of kidney injury molecule 1 (KIM-1) in a urine sample from the subject,
comparing the amounts of L-FABP and KIM-1 determined with reference amounts of L-FABP and KIM-1,
calculating an L-FABP/KIM-1 ratio from the amounts determined and comparing the calculated ratio with a reference L-FABP/KIM-1 ratio,
determining an amount of N-terminal pro brain natriuretic peptide (NT-proBNP) in a serum sample from the subject,
comparing the amount of NT-proBNP determined with a reference amount of NT-proBNP,
using the comparisons made to monitor the kidney damage in the subject.

8. A device for diagnosing kidney damage in a subject with heart failure or suspected to suffer from heart failure, the device comprising:
means for determining an amount of liver-type fatty acid binding protein (L-FABP) and an amount of kidney injury molecule 1 (KIM-1) in a sample from the subject,
means for comparing the amounts of L-FABP and KIM-1 determined with reference amounts of L-FABP and KIM-1,
whereby the device is adapted for diagnosing the kidney damage.

9. A kit for diagnosing kidney damage in a subject with heart failure or suspected to suffer from heart failure, the kit comprising:
reagents for determining an amount of liver-type fatty acid binding protein (L-FABP) and an amount of kidney injury molecule 1 (KIM-1) in a sample from the subject, and
instructions for comparing the amounts of L-FABP and KIM-1 determined with reference amounts of L-FABP and KIM-1 whereby a diagnosis of kidney damage may be made.
